(19) United States
(12) Patent Application Publication
(10) Pub. No.: US 2023/0240783 A1
(43) Pub. Date: Aug. 3, 2023

(54) Remote Monitoring of Disposal Bins for Reprocessable Medical Devices

(71) Applicant: DeRoyal Industries, Inc., Powell, TN (US)

(72) Inventors: Gregory S. Hodge, Knoxville, TN (US); Joe L. Smith, Powell, TN (US)

(73) Assignee: DeRoyal Industries, Inc., Powell, TN (US)

(21) Appl. No.: 18/104,820

(22) Filed: Feb. 2, 2023

Related U.S. Application Data
(60) Provisional application No. 63/306,149, filed on Feb. 3, 2022.

(51) Int. Cl. A61B 50/36; G16H 40/40; G16H 40/67

(52) U.S. Cl. CPC A61B 50/36; G16H 40/40; G16H 40/67

(57) ABSTRACT

An apparatus monitors a collection bin in which used medical devices are deposited during a medical procedure. The apparatus includes a dolly on which the collection bin is disposed, a sensor that senses whether the collection bin is full of reprocessable medical devices, a communication device that generates and sends an alert message via a communication network when the collection bin is full, and a visual indicator that generates a visual alert based on the sensor signal, wherein the visual alert indicates that the reprocessable medical devices in the collection bin should be sent for reprocessing. Based on receipt of the alert message transmitted via the communication network, a procedure is implemented for collecting and reprocessing the reprocessable medical devices in the collection bin.

### RELATED APPLICATIONS

This application claims priority to provisional patent application serial number 63/306,149, filed Feb. 3, 2022, titled Remote Monitoring of Disposal Bins for Reprocessable Medical Devices, the entire contents of which are incorporated herein by reference.

### FIELD

This invention relates to the field of medical devices. More particularly, this invention relates to a system for remotely monitoring collection bins in a medical facility and reporting when the bins are ready to be picked up for delivery of reusable devices in the bins to a reprocessing facility.

### BACKGROUND

Orthopedic surgical procedures at ambulatory surgery centers sometimes involve the use of valuable medical devices that are generally thought of as disposable, also referred to single-use devices. Although single-use devices may be disposable, they may be reused in other procedures if they are properly reprocessed. After use, these reusable devices are often collected in collection bins in the procedure room. The collection bins should be monitored to determine when it is time to send the devices out for reprocessing. It is time-consuming and expensive for medical personnel to do such monitoring.

What is needed, therefore, is an automated method for remotely monitoring disposal bins to determine when the devices disposed therein should be picked up for reprocessing.

### SUMMARY

The above and other needs are met by an apparatus that includes a dolly on which a collection bin is disposed, a sensor, and a visual indicator. The collection bin receives reprocessable medical devices after their use during the medical procedure. The sensor senses a state of the collection bin indicating that the collection bin is full or nearly full of reprocessable medical devices and generates a sensor signal based thereon. The visual indicator generates a visual alert based on the sensor signal, wherein the visual alert indicates that the reprocessable medical devices in the collection bin should be sent for reprocessing.

In some embodiments, the visual indicator comprises an indicator light disposed on the dolly.

In some embodiments, the visual indicator comprises a message displayed on a mobile communication device of medical personnel tasked with monitoring the state of the collection bin.

In some embodiments, the collection bin is removable from the dolly.

In some embodiments, the sensor and the visual indicator are integrated into the dolly.

In some embodiments, the apparatus includes a communication device that generates an alert message based on the sensor signal and sends the alert message via a wired or wireless connection through a communication network.

In another aspect, embodiments of the invention are directed to an apparatus for monitoring collection bins in which used reprocessable medical devices are deposited during a medical procedure. In one embodiment, the apparatus includes a collection bin, a sensor, and a communication device. The collection bin receives the reprocessable medical devices after their use during the medical procedure. The sensor senses a state of the collection bin indicating that the collection bin is full or nearly full of reprocessable medical devices and generates a sensor signal based thereon. The communication device generates an alert message based on the sensor signal and sends the alert message via a wired or wireless connection through a communication network.

In some embodiments, the apparatus includes a visual indicator that provides a visual alert indicating that the reprocessable medical devices in the collection bin should be sent for reprocessing. The visual indicator may be an indicator light disposed on the collection bin, or a message displayed on a mobile communication device of medical personnel tasked with monitoring the state of the collection bin.

In some embodiments, the sensor, the communication device, and the visual indicator are integrated into the collection bin.

In some embodiments, the apparatus includes a dolly upon which or in which the collection bin is disposed, wherein the collection bin may be removable from the dolly.

In some embodiments, a visual indicator associated with the dolly provides a visual alert indicating that the reprocessable medical devices in the collection bin should be sent for reprocessing.

In some embodiments, the sensor, the communication device, and the visual indicator are integrated into the dolly.

In yet another aspect, embodiments of the invention are directed to a method for monitoring a collection bin in which used reprocessable medical devices are deposited after their use during a medical procedure. The method includes:
sensing a state of the collection bin indicating that the collection bin is full or nearly full of reprocessable medical devices, and generating a state signal based on the state;
comparing the state signal to a threshold value;
based on the state signal exceeding the threshold value, generating an alert message;
transmitting the alert message via a communication network; and
based on receipt of the alert message via the communication network, implementing a procedure for collecting and reprocessing the reprocessable medical devices in the collection bin.

In some embodiments, the state of the collection bin is a weight of the collection bin, and the state signal is a weight signal.

In some embodiments, the method includes generating a visual alert based on the state signal, wherein the visual alert indicates that the procedure for collecting and reprocessing the reprocessable medical devices in the collection bin should be implemented.

In some embodiments, the visual indicator includes one or more of:
an indicator light viewable by medical personnel tasked with monitoring the state of the collection bin; and
a message displayed on a mobile communication device of the medical personnel tasked with monitoring the state of the collection bin.

In some embodiments, the method includes beginning a procedure to reorder and restock the reprocessable medical devices based on the alert message.

### BRIEF DESCRIPTION OF THE DRAWINGS

Other embodiments of the invention will become apparent by reference to the detailed description in conjunction with the figures, wherein elements are not to scale so as to show the details more clearly, wherein like reference numbers indicate like elements throughout the several views, and wherein:

FIG. 1 depicts a system for remote monitoring of collection bins for reprocessable medical devices according to an embodiment of the invention; and

FIG. 2 depicts a method for remote monitoring of collection bins for reprocessable medical devices according to an embodiment of the invention.

### DETAILED DESCRIPTION

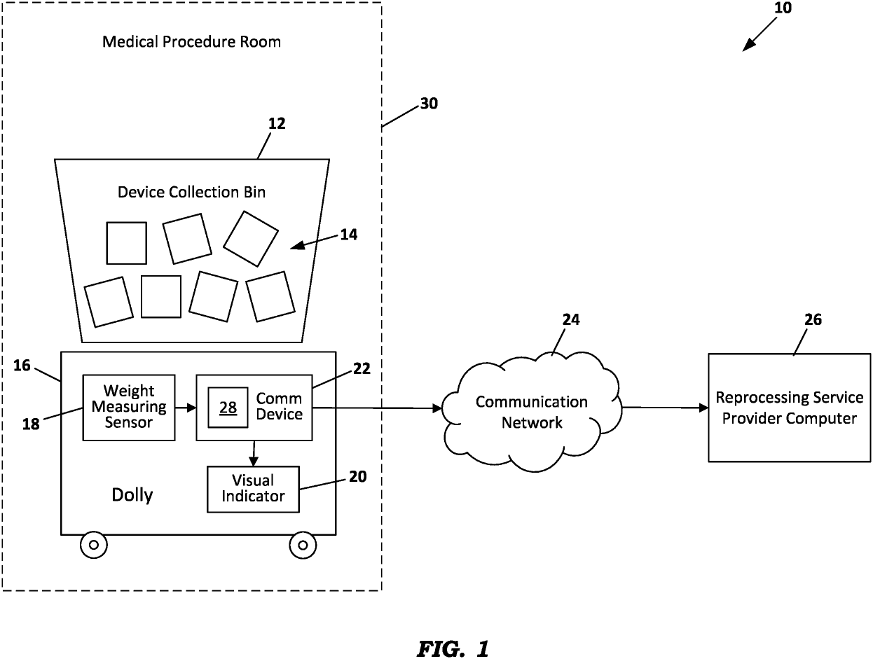

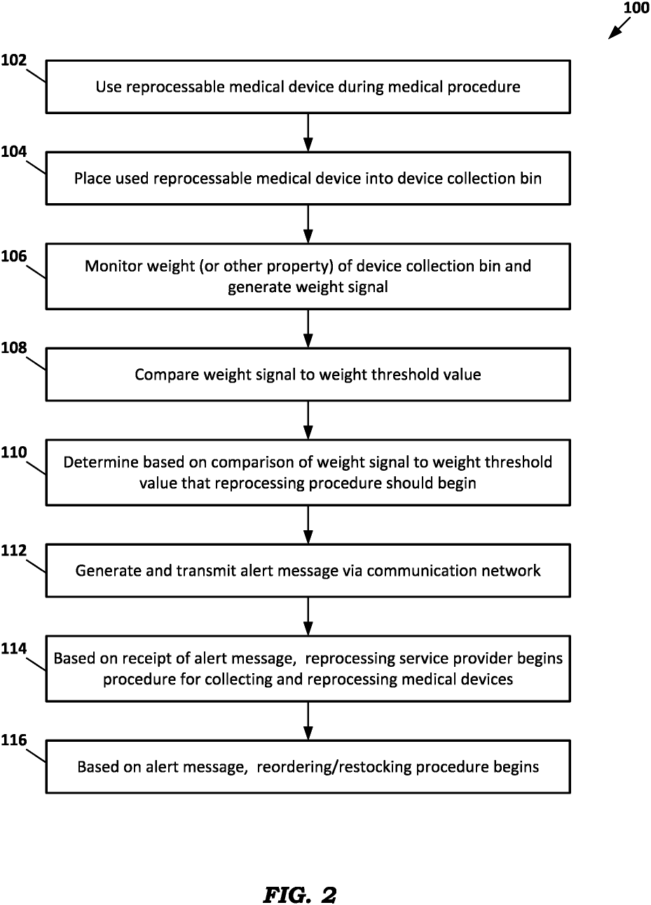

A system 10 for remote monitoring of collection bins for reprocessable medical devices is depicted in FIG. 1 and a method 100 for its use is depicted in FIG. 2.

After a reprocessable medical device 14 has been used during a medical procedure performed in a procedure room 30 (step 102 in FIG. 2), it is placed in a device collection bin 12 that sits atop or within a dolly 16 (step 104). Integrated into or attached to the dolly is a weight measuring sensor 18 that monitors the weight of the collection bin 12. The weight measuring sensor 18 generates a weight signal that is provided to a communication device 22 integrated into or attached to the dolly 16 (step 106). Based on comparison of the weight signal to a predetermined weight threshold value (step 108), a processor in the communication device 22 determines that the bin 12 is full or nearly full (step 110). The weight threshold value is preferably configurable by the user.

When the bin 12 is full or nearly full, the communication device 22 generates an alert message that is transmitted via a wired or wireless connection through a communication network 24, such as the Internet (step 112). Also connected to the communication network 24 is a computer 26 of a reprocessing service provider, which is an entity tasked with collecting and reprocessing the used medical devices 14 in the collection bin 12. Upon receipt of the alert message, the reprocessing service provider initiates a procedure for collecting and reprocessing the medical devices 14 in the collection bin 12 (step 114). The alert message may also indicate that the medical devices 14 need to be reordered to replenish stock, in which case the alert message is communicated to an entity responsible for reordering/restocking the medical devices (step 116).

It will be appreciated that the processor 28 is not necessarily a component of the communication device 22, as depicted in FIG. 1. The processor 28 could alternatively be a component of the weight measuring sensor 18, or a stand-alone component within the dolly 16. It should also be appreciated that the sensor 18, processor 28, and communication device 22 could be integrated into the collection bin 12 rather than into the dolly 16.

A visual indicator 20, such as a flashing LED on the dolly 16, may also be activated when the alert message is generated. This provides a visual alert to personnel in the operating room 30 that the medical devices 14 in the collection bin 12 should be sent for reprocessing and replacement devices should be ordered.

In addition to the visual indicator 20, other alert technologies may be implemented to alert personnel that are in or near the location of the collection bin 12, such as using WiFi, Bluetooth, and/or cellular data communications to communicate with mobile communication devices of personnel tasked with monitoring the status of collection bins 12.

In alternative embodiments, properties other than or in addition to weight may be monitored to determine that the collection bin 12 is full or nearly full. For example, such additional properties may be sensed by one or more of the following sensors:
a camera for monitoring the interior of the bin;
a time-of-flight (TOF) sensor for monitoring the distance from the top of the bin to the top of a pile of used medical devices in the bin;
an RFID sensor for sensing and counting RFID tags on used medical devices in the bin;
a sensor on a lid of the bin, wherein the lid must be opened to deposit used medical devices therein, and the sensor determines how many times the lid is opened;
an arm or motion sensor within the bin for detecting when each used medical device is deposited therein; and
a sound or vibration sensor for detecting each used medical device being deposited into the bin.

Any or all of the above-listed sensor technologies may be integrated into the collection bin 12 rather than into the dolly 16. One or more of the sensors may be in the bottom of the bin, at the top of the bin, beside the bin, or surrounding the bin.

The foregoing description of preferred embodiments for this invention have been presented for purposes of illustration and description. They are not intended to be exhaustive or to limit the invention to the precise form disclosed. Obvious modifications or variations are possible in light of the above teachings. The embodiments are chosen and described in an effort to provide the best illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. An apparatus for monitoring collection bins in which used reprocessable medical devices are deposited during a medical procedure, the apparatus comprising:
a collection bin for receiving the reprocessable medical devices after use during the medical procedure;
a sensor that senses a state of the collection bin indicating that the collection bin is full or nearly full of reprocessable medical devices, and that generates a sensor signal based thereon; and
a communication device that generates an alert message based on the sensor signal and sends the alert message via a wired or wireless connection through a communication network.

2. The apparatus of claim 1 further comprising a visual indicator that provides a visual alert indicating that the reprocessable medical devices in the collection bin should be sent for reprocessing.

3. The apparatus of claim 2 wherein the visual indicator comprises an indicator light disposed on the collection bin.

4. The apparatus of claim 2 wherein the visual indicator comprises a message displayed on a mobile communication device of medical personnel tasked with monitoring the state of the collection bin.

5. The apparatus of claim 1 wherein the sensor and the communication device are integrated into the collection bin.

6. The apparatus of claim 1 further comprising a dolly upon which or in which the collection bin is disposed.

7. The apparatus of claim 6 wherein the collection bin is removable from the dolly.

8. The apparatus of claim 6 further comprising a visual indicator associated with the dolly that provides a visual alert indicating that the reprocessable medical devices in the collection bin should be sent for reprocessing.

9. The apparatus of claim 6 wherein the sensor and the communication device are integrated into the dolly.

10. An apparatus for monitoring collection bins in which used reprocessable medical devices are deposited during medical procedures, the apparatus comprising:
a dolly that is configured to receive a collection bin in which reprocessable medical devices are disposed after use during a medical procedure;
a sensor associated with the dolly that senses a state of the collection bin indicating that the collection bin is full or nearly full of reprocessable medical devices, and that generates a sensor signal based thereon; and
a visual indicator associated with the dolly that generates a visual alert based on the sensor signal, wherein the visual alert indicates that the reprocessable medical devices in the collection bin should be sent for reprocessing.

11. The apparatus of claim 10 wherein the visual indicator comprises an indicator light disposed on the dolly.

12. The apparatus of claim 10 wherein the visual indicator comprises a message displayed on a mobile communication device of medical personnel tasked with monitoring the state of the collection bin.

13. The apparatus of claim 10 wherein the collection bin is removable from the dolly.

14. The apparatus of claim 10 wherein the sensor and the visual indicator are integrated into the dolly.

15. The apparatus of claim 10 further comprising a communication device associated with the dolly that generates an alert message based on the sensor signal and sends the alert message via a wired or wireless connection through a communication network.

16. A method for monitoring a collection bin in which used reprocessable medical devices are deposited after their use during a medical procedure, the method comprising:
(a) sensing a state of the collection bin indicating that the collection bin is full or nearly full of the reprocessable medical devices, and generating a state signal based on the state;
(b) comparing the state signal to a threshold value;
(c) based on the state signal exceeding the threshold value, generating an alert message;
(d) transmitting the alert message via a communication network; and
(e) based on receipt of the alert message via the communication network, implementing a procedure for collecting and reprocessing the reprocessable medical devices in the collection bin.

17. The method of claim 16 wherein the state of the collection bin is a weight of the collection bin, and the state signal is a weight signal.

18. The method of claim 16 further comprising generating a visual alert based on the state signal, wherein the visual alert indicates that the procedure for collecting and reprocessing the reprocessable medical devices in the collection bin should be implemented.

19. The method of claim 18 wherein the visual indicator comprises one or more of:
an indicator light viewable by medical personnel tasked with monitoring the state of the collection bin; and
a message displayed on a mobile communication device of medical personnel tasked with monitoring the state of the collection bin.

20. The method of claim 16 further comprising beginning a procedure to reorder and restock the reprocessable medical devices based on the alert message.
